(19) United States
(12) Patent Application Publication
(10) Pub. No.: US 2015/0119401 A1
(43) Pub. Date: Apr. 30, 2015

(54) Compositions and Methods for the Treatment of Nasal Conditions

(71) Applicant: Eye Therapies, LLC, Dana Point, CA (US)

(72) Inventor: Gerald Horn, Deerfield, IL (US)

(21) Appl. No.: 14/593,235

(22) Filed: Jan. 9, 2015

Related U.S. Application Data
(63) Continuation-in-part of application No. 14/010,340, filed on Aug. 26, 2013, now Pat. No. 8,952,011.

(51) Int. Cl. A61K 31/498; A61K 47/32; A61K 47/38

(52) U.S. Cl. CPC A61K 31/498; A61K 47/38; A61K 47/32
USPC 514/249

(57) ABSTRACT

The invention provides compositions and methods for treating nasal congestion. The provided compositions and methods utilize low concentrations of selective a-2 adrenergic receptor agonists. The compositions preferably include brimonidine.

### CROSS-REFERENCE TO RELATED APPLICATIONS

This application is a continuation-in-part of U.S. patent application Ser. No. 14/010,340 filed Aug. 26, 2013, which is a continuation-in-part of U.S. patent application Ser. No. 13/066,370 filed Apr. 13, 2011, which is a continuation-in-part of U.S. patent application Ser. No. 12/460,967 filed Jul. 27, 2009, which claims a priority to U.S. Provisional Application Ser. Nos. 61/137,714, filed on Aug. 1, 2008; 61/192,777, filed on Sept. 22, 2008; 61/203,120, filed on Dec. 18, 2008; and 61/207,481 filed on Feb. 12, 2009. The contents of the above-mentioned applications are hereby incorporated by reference in their entirety.

### BACKGROUND OF THE INVENTION

Adrenergic receptors mediate physiological responses to the catecholamines, norephinephrine and epinephrine, and are members of the superfamily of G protein-coupled receptors having seven transmembrane domains. These receptors, which are divided pharmacologically into α-1, α-2 and β-adrenergic receptor types, are involved in diverse physiological functions including functions of the cardiovascular and central nervous systems. The α-adrenergic receptors mediate excitatory and inhibitory functions: α-1 adrenergic receptors are typically excitatory post-synaptic receptors which generally mediate responses in an effector organ, while α-2 adrenergic receptors are located postsynaptically as well as presynaptically, where they inhibit release of neurotransmitters. The α-adrenergic receptors also mediate vascular constriction. Agonists of α-2 adrenergic receptors currently are used clinically in the treatment of hypertension, glaucoma, spasticity, and attention-deficit disorder, in the suppression of opiate withdrawal, as adjuncts to general anesthesia and in the treatment of cancer pain.

α-2 adrenergic receptors are present in various bodily organs, including eyes and nose. It is believed that they play a role in nasal congestion, among many other diseases.

α-2 adrenergic receptors are presently classified into three subtypes based on their pharmacological and molecular characterization: α-2A/D (α-2A in human and α-2D in rat); α-2B; and α-2C (Bylund et al., Pharmacol. Rev. 46:121-136 (1994); and Hein and Kobilka, Neuropharmacol. 34:357-366 (1995)). The α-2A, α-2B, and α-2C subtypes appear to regulate arterial and/or venular contraction in some vascular beds, and the α-2A and α-2C subtypes mediate feedback inhibition of norepinephrine release from sympathetic nerve endings.

Many compounds having selective α-2 agonist activity are known and include brimonidine (which has been used for lowering intraocular pressure in patients with open-angle glaucoma or ocular hypertension), guanfacine (which has been used to control high blood pressure), dexmedetomidine (which has been used as a sedative, analgesic, sympatholytic and anxiolytic), and methyl dopa (which has been used as a centrally -acting adrenergic antihypertensive).

Nasal conditions, such as nasal congestion, cause inconvenience and suffering to many individuals. The use of conventional decongestant nasal sprays cause rebound congestion, often lasting 24 hours or longer, which typically results after using these sprays for more than three consecutive days, or even after a single day's use. In addition, continued use of conventional nasal decongestants (such as Afrin®, Afrin is a registered trademark of MSD Consumer Care, Inc.; Dristan®, Dristan is a registered trademark of Wyeth LLC; and many others) may result in chronic and long term inflammatory pathological conditions. These conditions frequently occur as a subject attempts to reverse the rebound congestion with more and more frequent use of the conventional nasal decongestant. Phenylephrine, a strong α-1 agonist, and oxymetazoline, a strong α-1 agonist with some α-2 agonist activity, are powerful nasal decongestants. However, these decongestants are associated with numerous side effects upon repeat use. Rhinitis medicamentosa is one such result side effect that results from inflammatory ischemic changes caused by such patterns of use. Rhinitis medicamentosa ultimately results in a total nasal blockage which may not be relieved by simply stopping the medication. It may take days, weeks, months, or even medical or surgical intervention to treat rhinitis medicamentosa. It is currently estimated that 10 million people in the U.S. alone suffer from this condition.

It is a long held dogma of prior art that all topical α-agonists when used nasally induce vasoconstriction, and as a result, cause ischemia. Thus, it is thought that all topical α-agonists, when repeatedly topically applied to mucosal surfaces, result in rebound hyperemia and/or congestion, tachyphylaxis, and chronic ischemic inflammatory change, such as rhinitis medicamentosa.

Thus, there is a need in the art for new compositions and methods that would be useful for treatment of nasal conditions, including but not limited to nasal congestion, which cause long lasting relief with no or only transient (i.e., only a few hours, with very low incidence) rebound congestion and no rhinitis medicamentosa. There is also a need for new formulations for medications useful for the treatment of nasal congestion, whereby said medications can be administered through the nasal route to relieve nasal congestion on a regular basis without significant rebound congestion and/or rhinitis medicamentosa.

### SUMMARY OF THE INVENTION

The present invention provides compositions and methods for treating a nasal condition by administering low concentrations of highly selective α-2 adrenergic receptor agonists to a subject in need thereof.

The provided compositions and methods utilize low concentrations of highly selective α-2 adrenergic receptor agonists having a binding affinity of 100 fold or greater for α-2 over α-1 adrenergic receptors. The concentration of the selective α-2 adrenergic receptor agonist is preferably below the concentration at which α-1 adrenergic receptors are activated sufficiently enough to cause adverse ischemic vasoconstrictive consequences. Preferably, the concentration of the selective α-2 adrenergic receptor agonist is below about 0.05% weight by volume of the composition.

In preferred embodiments of the invention, the selective α-2 adrenergic receptor agonist is selected from the group consisting of lofexidine, apraclonidine, mivazerol, brimonidine, alpha methyl dopa, guanfacine, fadolmidine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds.

In a preferred embodiment, a pH of the composition comprising the selective α-2 adrenergic receptor agonist is between about 4.0 and about 8.5. If it is desired to achieve a more effective topical mucosal application with minimal mucosal penetration (for example, in such conditions as vasomotor rhinitis or nasal congestion), then it is generally preferred to maintain pH of the composition between about 4.0 and about 6.5. If, on the other hand, it is desired to achieve a deeper mucosal penetration (for example, delivery of drugs intravascularly), then a preferred pH of the composition may be between about 6.0 and about 8.0.

If more prolonged effect is desired nasally than two to three sprays per naris about one minute apart may be preferred, each spray optionally directed from tip to mid turbinate to superior turbinate. If delivery of central nervous system drug particularly a drug directed to the cerebral cortex is desired than delivery primarily to the superior turbinate via deeper penetration of the tip into the nares may be preferred.

In one embodiment of the invention, the compositions of the invention can be administered by nasal delivery. In another embodiment of the invention, the compositions of the invention can be administered by topical ophthalmic delivery.

In a preferred embodiment, the invention provides a composition comprising from about 0.01% to about 0.04% w/v brimonidine and a vehicle selected from one or more cellulosic vehicles and polyvinylpyrrolidone or a combination thereof; more preferably 0.035% w/v brimonidine.

In a more preferred embodiment, the one or more cellulosic vehicles are selected from carboxymethyl cellulose, microcrystalline cellulose, a mixture of carboxymethyl cellulose and microcrystalline cellulose, hydroxypropyl cellulose and hydroxpropylmethyl cellulose.

In another preferred embodiment, the invention provides a composition comprising from about 0.01% to about 0.04% w/v brimonidine and a vehicle comprising a mixture of carboxymethyl cellulose and microcrystalline cellulose, polyvinylpyrrolidone and hydroxypropyl cellulose.

In another more preferred embodiment, the concentration of hydroxypropyl cellulose is from about 0.96% to about 1.36% w/v, most preferably about 1.167% w/v.

In another preferred embodiment, the invention provides a composition comprising from about 0.01% to about 0.04% w/v brimonidine and a vehicle comprising a mixture of carboxymethyl cellulose and microcrystalline cellulose, polyvinylpyrrolidone and hydroxypropylmethyl cellulose.

In another more preferred embodiment, the concentration of hydroxypropylmethyl cellulose is from about 2% to about 4% w/v, most preferably about 3% w/v.

In another preferred embodiment the present invention provides a composition comprising:
brimonidine at an amount of about 0.035% w/v;
3.0% w/v of a coprecipitate consisting of carboxymethyl cellulose and microcrystalline cellulose;
polyvinylpyrrolidone at an amount of about 3.0% w/v; and
hydroxypropyl cellulose at an amount of about 1.167% w/v.

In another embodiment of the invention, the composition further comprises pharmaceutically acceptable excipients selected from the group consisting of preservatives, tonicity adjustors, pH adjustors and permeation enhancers.

In another embodiment, the invention is directed to a method of treating a nasal condition including but not limited to insufficient nares patency for peak athletic performance, rhinitis medicamentosa secondary to oxymetazoline nasal spray, allergic rhinitis, vasomotor rhinitis, sleep apnea, nasal secretion induced gastroesophogeal reflux, sleep apnea due to obstructed or partially obstructed turbinates, and treatment of partial or complete nasal obstruction due to nasal polyps or a combination thereof comprising administering to a subject in need thereof a pharmaceutically effective amount of a composition of the invention, wherein the subject experiences minimal or no rebound.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1 is a graphical representation of the effects of activating α-1 adrenergic receptors; and

FIG. 2 is a graphical representation of the effects of preferentially activating α-2 adrenergic receptors.

### DETAILED DESCRIPTION OF THE INVENTION

### Definitions

For purposes of the present invention, the terms below are defined as follows.

The term “low concentrations” refers to concentrations from between about 0.0001% to about 0.065%; more preferably, from about 0.001% to about 0.035%; even more preferably, from about 0.01% to about 0.035%; and even more preferably, from about 0.030% to about 0.035% weight by volume of the composition.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan® (Alphagan is a registered trademark of Allergan, Inc.), and UK14304.

The terms “treating” and “treatment” refer to reversing, alleviating, inhibiting, or slowing the progress of the disease, disorder, or condition to which such terms apply, or one or more symptoms of such disease, disorder, or condition.

The term “nasal condition” refers to any disease, disorder, or condition which affects and/or involves the nose. This term includes, but is not limited to, such conditions as nasal congestion, diseases and/or conditions associated with swollen nasal turbinates, all types of rhinitis including but not limited to vasomotor rhinitis and allergic rhinitis, sleep apnea, acute or chronic sinusitis, nasal polyposis, and any disease and/or condition associated with nasal discharge.

The term “substantial enlargement of nasal turbinates” refers to a significant enlargement of nasal turbinates, for example, more than about 50% compared to the baseline level of the subject so that it negatively affects the subject's breathing.

The term “subject” refers but is not limited to a person or other animal.

The term “over-the-counter” refers to components that can and have been used in pharmaceutical compositions that are sold in the United States without a prescription, because they have applied for and been approved for such use by the Food and Drug Administration or are exempt from the application process because they are generally recognized as safe and effective by medical professionals.

The term “rebound” refers, but is not limited to, the reoccurrence or worsening of symptoms including congestion and hyperemia.

The term “% w/v” refers to the percent weight by volume of the total composition. Unless otherwise noted all percentages in the instant application refer to weight by volume.

### Embodiments of the Invention

It was surprisingly and unexpectedly found that selective alpha-2 (α-2) adrenergic receptor agonists (which are interchangeably referred to as “α-2 agonists” throughout the application) with extremely high selectivity for α-2 adrenergic receptors (meaning selectivity of at least 500:1 for α-2 over α-1 adrenergic receptors and preferably at least 900:1 cumulatively for α-2 subtypes A, B and C, and preferably predominantly for at least two of the three subtypes) at sufficiently low concentrations and at pH of between about 4.0 and about 8.5 can be used to treat a nasal condition in a subject in need thereof.

One example of a nasal condition is turbinate mucosal swelling which is caused by, or is contributed by, vasodilation and leakage of blood vessels. While not wishing to be bound to any particular theory, it is believed that vasodilation is after a short period of intense vasoconstriction of large and small arterioles associated with induced ischemia and inflammation inherent to such pervasive constriction consequent to α-1 adrenergic receptors activity. This α-1 effect is so dominant and fundamental to α-1 receptor activity it has been discovered via the present invention to occur not only after predominant α-1 agonist use, such as topical phenylephrine or tetrahydrozoline, but with α-2 agonists unless the binding affinity of α-2 agonists for α-2 over α-1 adrenergic receptors is sufficiently high, and the concentration sufficiently low to limit the pool of α-1 receptors inadvertently triggered. Otherwise, insufficiently highly selective α-2 agonists cause undesirable proportions of α-1 receptor stimulation with attendant ischemic vasoconstriction, proinflammatory cytokine release, and rebound vasodilation with repeat use such as is commonly found with agonists such as oxymetazoline.

Accordingly, the invention is directed to compositions and methods which employ highly selective α-2 agonists that have minimal α-1 agonist activity at extremely low concentrations, where for example 1% to 2% is considered extremely high, 0.5% to 1.0% still highly inductive of α-1 receptors and toxic for purposes of the present invention, 0.10% to 0.5% still too high, 0.070% to 0.10% still associated with a higher than preferred incidence of rebound, and only 0.065% or below potentially acceptable, where for most agonists, depending on degree of selectivity 0.050% or even more preferably 0.035% or less is desired. On the other hand some degree of useful activity may occur at one or more orders of magnitude further reduction of concentration. The compositions of the present invention preferentially stimulate α-2 adrenergic receptors so that α-1 adrenergic receptors are not stimulated sufficiently enough to cause vasodilation.

Thus, in one embodiment, the invention provides a method of treating a nasal condition comprising administering to a subject in need thereof a selective α-2 adrenergic receptor agonist having a binding affinity of 100 fold or greater for α-2 over α-1 adrenergic receptors, or a pharmaceutically acceptable salt thereof, wherein said selective α-2 adrenergic receptor agonist is present at a concentration below about 0.05% weight by volume.

In one embodiment, the invention provides compositions formulated for treating a condition associated with swollen nasal turbinates. Compositions particularly useful for these purposes preferably comprise brimonidine at concentrations of from about 0.01% to about 0.04%, more preferably, from about 0.025% to about 0.04%, even more preferably, from about 0.03% to about 0.0375%, and most preferably from about 0.03% to about 0.035%. In a preferred embodiment, the condition associated with swollen nasal turbinates is selected from the group consisting of nasal congestion, allergic rhinitis, asthma, sleep disorders, and sleep apnea. A preferred pH of the composition formulated for the condition associated with swollen nasal turbinates is between about 6.5 and about 8.5.

### Selective α-2 Adrenergic Receptor Agonists Suitable for the Purposes of the Invention

Selective α-2 agonists that may be used for the purposes of the present invention have extremely high selectivity for α-2 adrenergic receptors, defined by their binding affinities (Kᵢ) for α-2 over α-1 receptors of more than 100:1, more preferably 300:1; more preferably 500:1, even more preferably 700:1, even more preferably about 1000:1 or greater, and most preferably, 1500:1 or greater, where the ultimate preference may vary depending on the receptor mix of α-2 subtype activity for the particular species of mammal, and even further the ethnicity. In most cases subtypes A and B binding activity will be preferred over C for relief of local congestion; whereas subtype C binding activity may provide added value in some patients particularly for cerebral applications (e.g. migraine).

Not desiring to be bound by any specific theory or mechanism, it is believed that the particularly preferred adrenergic receptor agonists for most of the purposes of the present invention are highly selective for α-2B and/or α-2C receptors, as opposed to α-2A receptors.

In one embodiment, the selective α-2 adrenergic receptor agonist is a compound which has binding affinity of about 100 fold or greater for α-2 over α-1 adrenergic receptors, preferably about 300 fold or greater, more preferably about 700 fold or greater, even more preferably about 1000 fold or greater, and most preferably, about 1500 fold or greater.

The selective α-2 adrenergic receptor agonist may be present at a concentration from between about 0.0001% to about 0.05%; more preferably, from about 0.001% to about 0.035%; even more preferably, from about 0.01% to about 0.035%; and even more preferably, from about 0.03% to about 0.0375% weight by volume and most preferably from about 0.03% to about 0.035%.

It is preferred that a concentration of a selective α-2 adrenergic receptor agonist be below its vasoconstriction vs. concentration plateau. Typically, the optimal concentration is 10% to 90% above the minimal threshold of measurable vasoconstriction for a particular α-2 agonist, or below that of the plateau maximum concentration, and is preferably within the about 25% to about 75% range of either of these benchmarks. The term “plateau maximum concentration” means the concentration above which there is no or minimal further vasoconstriction effect. Other considerations in choosing a selective α-2 adrenergic receptor agonist are blood brain permeability and any possible side effects and other systemic reactions.

In one embodiment, the selective α-2 adrenergic receptor is selected from the group consisting of lofexidine, apraclonidine, mivazerol, brimonidine, alpha methyl dopa, guanfacine, fadolmidine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds. Analogs of these compounds that function as highly selective α-2 agonists may also be used in compositions and methods of the present invention.

In a more preferred embodiment, the selective α-2 adrenergic receptor is brimonidine in the form of a salt. In a preferred embodiment, the salt is tartrate salt.

### Compositions and Methods of the Invention

In one embodiment, the invention provides a composition comprising a selective α-2 adrenergic receptor agonist having a binding affinity of 100 fold or greater for α-2 over α-1 adrenergic receptors, or a pharmaceutically acceptable salt thereof, for treating nasal congestion.

In a preferred embodiment, said selective α-2 adrenergic receptor agonist is present at a concentration below about 0.05% weight by volume, and more preferably, between about 0.001% to about 0.05% weight by volume.

In one embodiment, the selective α-2 adrenergic receptor agonist is selected from the group consisting of lofexidine, apraclonidine, mivazerol, clonidine, brimonidine, alpha methyl dopa, guanfacine, fadolmidine, dexmedetomidine, (+)-(S)-4-[1-(2,3-dimethyl-phenyl)-ethyl] -1,3-dihydro-imidazole-2-thione, 1-[(imidazolidin-2-yl)imino]indazole, and mixtures of these compounds.

In a preferred embodiment, the composition comprises brimonidine at a concentration between about 0.001% and about 0.035% weight by volume.

In a preferred embodiment, a pH of the composition comprising the selective α-2 adrenergic receptor agonist is between about 4.0 and about 8.5.

If it is desired to achieve a more effective topical mucosal application with minimal mucosal penetration (for example, in such conditions as vasomotor rhinitis or nasal congestion with substantial nasal discharge but relatively minimal turbinate swelling or physical blockage of nasal passages), then it is generally preferred to maintain pH of the composition between about 4.0 and about 6.5.

There is a direct relationship between a selective α-2 agonist's lipophilicity (as characterized by the Log D value) and the pH of a pharmaceutical composition containing the selective α-2 agonist: as the pH increases across the range of 4.0 to 8.5, the selective α-2 agonist's nonionic versus ionic equilibrium shifts to the left, so that its lipophilicity exponentially increases. This correlation is true for virtually all selective α-2 agonists, and in particular, brimonidine and dexmedetomidine, where such shift is relative to the intrinsic starting point—the Log P value—the lipophilicity at about pH 7.4 of the particular α-2 agonist.

Log D refers to a lipophilicity value at a given pH. This measurement is especially useful to determine the level of topical lipophilicity and resultant permeability of a topical composition. The higher the lipophilicity, the greater is the selective α-2 agonist's penetration through the lipophilic mucosal epithelial cell membranes. This is because at a more alkaline pH, more of the compound is present in a non-ionized form. When the pH is relatively low, e.g. between about 4.0 and about 6.5, the selective α-2 agonist is relatively less lipophilic and more ionized. As a result, a greater percentage of the selective α-2 agonist remains on the mucosa, increasing the drug's effectiveness as compared to the results at a higher pH. Thus, pH range of 4.0 to about 6.5, and more preferably 4.0 to 5.8, is preferred for the formulations for the treatment of nasal conditions involving serous nasal discharge without substantial turbinate swelling, such as vasomotor rhinitis.

If, on the other hand, it is desired to achieve a deeper mucosal penetration (for example, in such conditions as allergic rhinitis or sleep apnea; and generally in any nasal condition involving substantial enlargement of the nasal turbinates and/or physical blockage of nasal passages), then a preferred pH of the composition is between about 6.5 and about 8.5. At this higher pH, a greater proportion of the α-2 agonist will be non- ionized and more lipophilic, resulting in the greater permeation of the α-2 agonist through the lipophilic mucosal epithelial cell membranes. Thus, pH range of 6.5 to 8.5, and more preferably, 7.5 to 8.5 is preferred for formulations for intravascular drug delivery and or disorders associated with need for greater penetration of nasal turbinates, such as substantial enlargement of nasal turbinates and/or physical blockage of nasal passages, for example due to venous sinusoidal dilation as may occur in more severe cases of allergic rhinitis or turbinate blockage associated with sleep apnea with or without nodules causing some or complete blockage absent the present invention.

For some nasal conditions, it may be preferred to achieve a moderate lipophilicity, which is associated with pH of between about 5.6 and 6.2.

Dexmedetomidine has the following Log D values at different pH:

pH 4.0 to 5.6: Log D is 0.76 to 1.76;

pH 5.6 to 6.2: Log D is 1.76 to 2.28;

pH 6.2 to 8.0: Log D is 2.28 to 3.00.

The lower the Log D value is, the less is lipophilicity and the more is surface retention and mucosal effectiveness. Conversely, the higher the Log D value is, the more is lipophilicity, and the more is mucosal penetration and submucosal permeation.

Brimonidine has the following Log D values at different pH:

pH 4.0 to 6.2: Log D is −1.02 to −0.44;

pH 7.0 to 8.0: Log D is 0.55 to 0.79.

When the selective α-2 agonist is brimonidine, the moderate lipophilicity is achieved at pH of between 6.2 and 6.8. A pH of less than 6.2 is preferred to achieve greater mucosal surface retention, and a pH of greater than 6.8 is preferred to achieve greater mucosal penetration and submucosal permeation.

In one embodiment, the invention provides an aqueous composition for treating a nasal condition consisting essentially of brimonidine, wherein said brimonidine concentration is from between about 0.03% to about 0.035% weight by volume, wherein pH of said composition is between about 6.2 and about 6.8.

In another embodiment, the compositions of the invention may also include additional components, which include, but are not limited to, preservatives, delivery vehicles, tonicity adjustors, buffers, pH adjustors, antioxidants, permeation enhancers and water.

Preservatives useful in a topical composition include, but are not limited to, benzalkonium chloride, chlorobutanol, thimerosal, phenylmercuric acetate, disodium ethylenediaminetetraacetic acid (“EDTA”), phenylmercuric nitrate, or benzyl alcohol. In a preferred embodiment of the invention the preservative consists of components that have been approved for use in over-the-counter (“OTC”) pharmaceutical compositions. In a more preferred embodiment the OTC suitable preservative is a mixture of disodium EDTA and benzyl alcohol. In a yet more preferred embodiment disodium EDTA is at an amount of from about 0.01% to about 0.1% w/v and benzyl alcohol is at an amount of from about 0.1% to about 1.0% w/v.

Vehicles useful in a topical composition include, but are not limited to, polyvinyl alcohol, polyvinylpyrrolidone, poloxamers, purified water and cellulosic vehicles including, but not limited to hydroxypropyl cellulose, hydroxypropylmethyl cellulose, carboxymethyl cellulose, hydroxyethyl cellulose or a mixture thereof. It is also possible to use a physiological saline solution as a major vehicle. In a preferred embodiment of the invention the vehicle consists of components that have been approved for use in OTC pharmaceutical compositions. In a more preferred embodiment the OTC suitable vehicle is a mixture of polyvinylpyrrolidone, carboxymethyl cellulose and hydroxypropyl cellulose or hydroxypropylmethyl cellulose. In a yet more preferred embodiment the carboxymethyl cellulose as a viscosity enhancer is supplemented by addition of a coprecipitate of carboxymethyl cellulose and microcrystalline cellulose; such that for a preferred embodiment the ratio of carboxymethyl cellulose to microcrystalline cellulose is from 9:91 to 13:87 - such as Avicel® 591 (Avicel is a registered trademark of FMC Corporation); where other ratios may be used, including but not limited to Avicel® RTM CL-611 or Avicel® RTM RC-581. Such viscosity supplementation as occurs with coprecipitates of carboxymethyl cellulose and microcrystalline cellulose provides enhanced thixotropic properties that it is believed adds stability to preferred embodiments and helps prevent sedimentation of ingredients, particularly during prolonged storage. In another more preferred embodiment the hydroxypropyl cellulose has a viscosity of about 2920 centipoise (1% in water at 25° C.). In yet another more preferred embodiment carboxymethyl cellulose is at an amount of from about 1.0% to about 5.0% w/v, polyvinylpyrrolidone is at an amount of from about 1.0% to about 5.0% w/v; hydroxypropyl cellulose is at an amount of from about 0% to about 5% w/v; more preferably from about 0.96% to about 1.36%, most preferably at about 1.167%; and hydroxypropylmethyl cellulose is from about 0% to about 5%, more preferably from about 2% to about 4%, most preferably at about 3%. In another embodiment hydroxpropyl cellulose is at a concentration of about 0.85% and hydroxypropylmethyl cellulose is at a concentration of 0.4%.

It is a further discovery of the present invention that the longest duration and efficacy is associated with a narrow range of brimonidine and viscosity, where the brimonidine range for the most preferred embodiments is from about 0.030% to about 0.375% in conjunction with a viscosity consistent with that found for hydroxypropyl cellulose at about 1.16% +/−0.2%; and where similarly other viscosity agents such as moderate molecular weight hydroxypropylmethyl cellulose create a similar benefit from about 3% to about 4%.

A tonicity adjustor also can be included, if desired, in a topical composition of the invention. Such a tonicity adjustor can be, without limitation, a salt such as sodium chloride, potassium chloride, mannitol or glycerin, or another pharmaceutically or ophthalmically acceptable tonicity adjustor. In a preferred embodiment of the invention the tonicity adjustor has been approved for use in OTC pharmaceutical compositions. In a more preferred embodiment the OTC suitable tonicity adjustor is glycerin. In a yet more preferred embodiment glycerin is at an amount of from about 0.1% to about 1.0% w/v.

Various buffers and means for adjusting pH can be used to prepare topical compositions of the invention. Such buffers include, but are not limited to, acetate buffers, citrate buffers, phosphate buffers and borate buffers. It is understood that acids or bases can be used to adjust the pH of the composition as needed. In a preferred embodiment of the invention the pH adjustor has been approved for use in OTC pharmaceutical compositions. In a more preferred embodiment the OTC suitable pH adjustor is a phosphate buffer. In a yet more preferred embodiment of the invention the phosphate buffer is a mixture of sodium phosphate, dibasic and sodium phosphate, monobasic. In yet another more preferred embodiment sodium phosphate, dibasic is at an amount of from about 0.01% to about 0.1% w/v and sodium phosphate, monobasic is at an amount of from about 0.1% to about 1.0% w/v.

Topically acceptable antioxidants useful in preparing a topical composition include, yet are not limited to, sodium metabisulfite, sodium thiosulfate, acetylcysteine, butylated hydroxyanisole and butylated hydroxytoluene.

Permeation enhancers that can be used in a topical composition of the present invention include, but are not limited to, menthol and polyethylene glycol-32 (“PEG-32”) or a mixture thereof. In a preferred embodiment of the invention the permeation enhancer consists of components that have been approved for use in OTC pharmaceutical compositions. In a more preferred embodiment the OTC suitable permeation enhancer is a mixture of menthol and PEG-32. In a yet more preferred embodiment PEG-32 is at an amount of from about 1.0% to about 10.0% w/v of and menthol is at an amount of from about 0.001% to about 0.1% w/v.

The compositions of the invention may be administered topically through nasal delivery or topically delivered as ophthalmic solutions into the eyes.

In one embodiment, the provided composition is an aerosolized composition. It is within a skill in the art to prepare aerosolized compositions of the present invention. The aerosolized compositions of the present invention are generally delivered via an inhaler, jet nebulizer, or ultrasonic nebulizer which is able to produce aerosol particles with size of between about 1 and 10 μm.

To make the topical compositions of the present invention, one can simply dilute, using methods known in the art, more concentrated solutions of selective α-2 agonists. The precise method of carrying out the dilutions is not critical. Any commonly used diluents, including preservatives described above in the application, suitable for topical solutions can be used.

Proper dosages of the compositions of the present invention are concentration-dependent. To determine the specific dose for a particular subject, a skilled artisan would have to take into account kinetics and absorption characteristics of the particular highly selective α-2 adrenergic receptor agonist.

The present invention is more fully demonstrated by reference to the accompanying drawings.

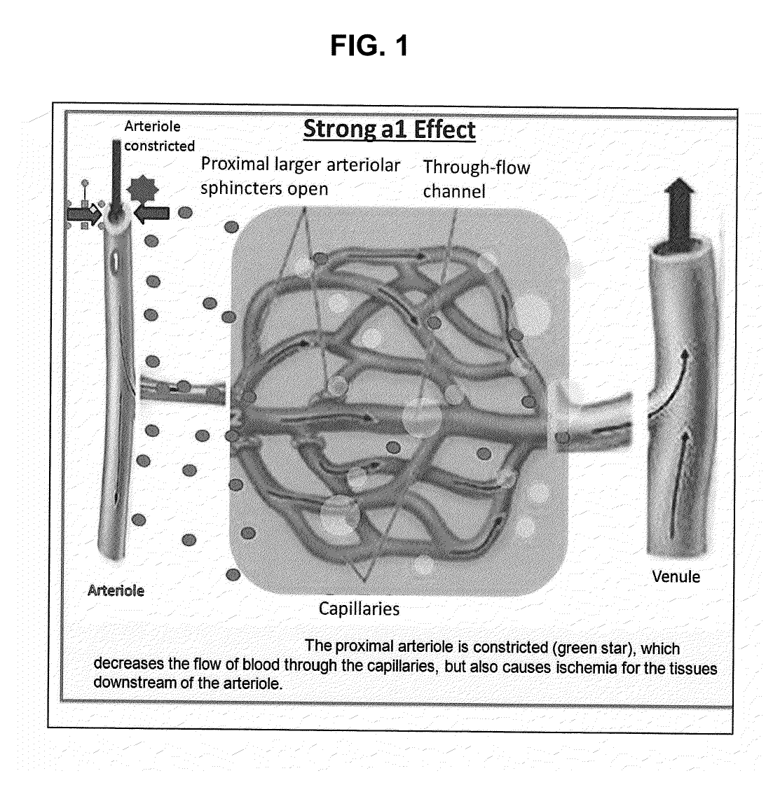

FIG. 1 is a graphical representation of the effects of activating α-1 adrenergic receptors. As FIG. 1 demonstrates, administering α-1 adrenergic receptor agonists leads to constriction of the proximal arteriole (on the left side) which in turn decreases the flow of blood through the capillaries and causes ischemia for the tissues downstream of arteriole.

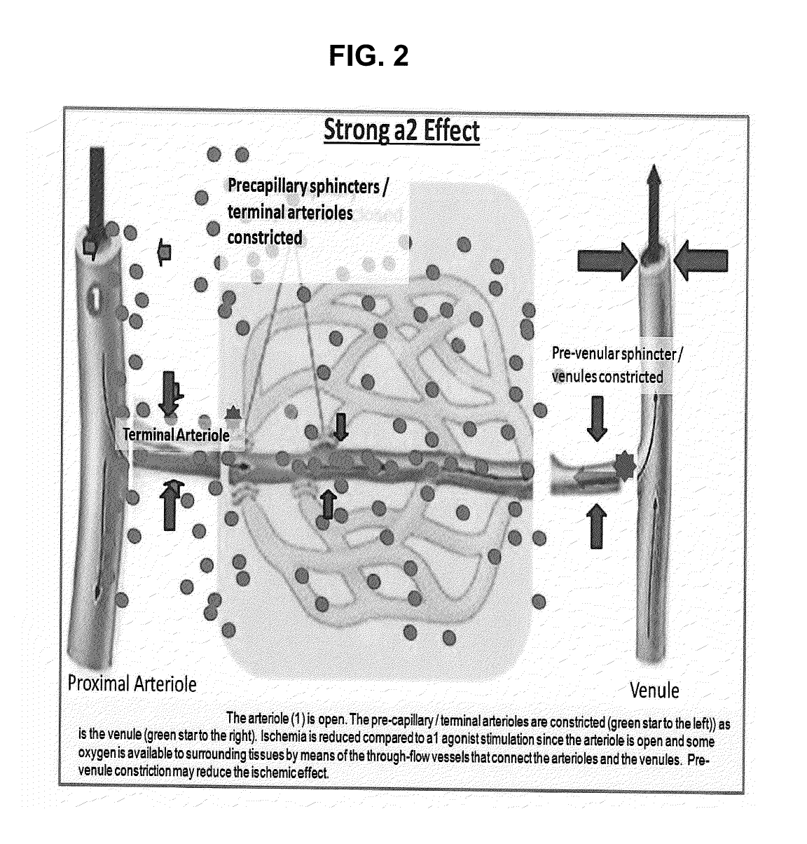

FIG. 2 is a graphical representation of the effects of preferentially activating α-2 adrenergic receptors. As FIG. 2 demonstrates, administering α-2 adrenergic receptor agonists leads to constriction of the pre-capillary/terminal arteriole (on the left side) and constriction of the venule (on the right side). Ischemia is decreased, as compared to stimulating α-1 adrenergic receptors because the arteriole is open and some oxygen is available to surrounding tissues by means of the through-flow vessels that connect the arterioles and the venules. Pre-venule constriction may reduce the ischemic effect and reduce vasodilation that may contribute to nasal congestion.

The following representative embodiments are provided solely for illustrative purposes and are not meant to limit the invention in any way. Additionally, the following representative embodiments each comprise brimonidine and a group of excipients that have been used in OTC pharmaceuticals.

### REPRESENTATIVE EMBODIMENTS

In a more preferred embodiment the composition comprises:

0.035% w/v brimonidine;

3.0% w/v Avicel® 591;

3.0% w/v polyvinylpyrrolidone;

5.0% w/v polyethylene glycol-32;

0.0975% w/v sodium phosphate, dibasic;

0.5525% w/v sodium phosphate, monobasic;

0.03% w/v disodium EDTA;

0.25% w/v benzyl alcohol;

0.5% w/v glycerin;

0.00375% w/v menthol; and

0.0% w/v hydroxypropyl cellulose.

In another more preferred embodiment the composition comprises:

0.035% w/v brimonidine;

3.0% w/v Avicel® 591;

3.0% w/v polyvinylpyrrolidone;

5.0% w/v polyethylene glycol-32;

0.0975% w/v sodium phosphate, dibasic;

0.5525% w/v sodium phosphate, monobasic;

0.03% w/v disodium EDTA;

0.25% w/v benzyl alcohol;

0.5% w/v glycerin;

0.00375% w/v menthol;

1.0% w/v hydroxypropyl cellulose; and

89.7229% w/v water.

In another more preferred embodiment the composition comprises:

0.035% w/v brimonidine;

3.0% w/v Avicel® 591;

3.0% w/v polyvinylpyrrolidone;

5.0% w/v polyethylene glycol-32;

0.0975% w/v sodium phosphate, dibasic;

0.5525% w/v sodium phosphate, monobasic;

0.03% w/v disodium EDTA;

0.25% w/v benzyl alcohol;

0.5% w/v glycerin;

0.00375% w/v menthol;

0.85% w/v hydroxypropyl cellulose; and

89.7229% w/v water.

In another more preferred embodiment the composition comprises:

0.035% w/v brimonidine;

3.0% w/v Avicel® 591;

3.0% w/v polyvinylpyrrolidone;

5.0% w/v polyethylene glycol-32;

0.0975% w/v sodium phosphate, dibasic;

0.5525% w/v sodium phosphate, monobasic;

0.03% w/v disodium EDTA;

0.25% w/v benzyl alcohol;

0.5% w/v glycerin;

0.00375% w/v menthol; and

2.0% w/v hydroxypropyl cellulose.

In a more preferred embodiment the composition comprises:

0.035% w/v brimonidine;

3.0% w/v Avicel® 591;

3.0% w/v polyvinylpyrrolidone;

5.0% w/v polyethylene glycol-32;

0.0975% w/v sodium phosphate, dibasic;

0.5525% w/v sodium phosphate, monobasic;

0.03% w/v disodium EDTA;

0.25% w/v benzyl alcohol;

0.5% w/v glycerin;

0.015% w/v menthol; and

2.0% w/v hydroxypropyl cellulose.

In another preferred embodiment the composition comprises:

0.035% w/v brimonidine;

3.0% w/v Avicel® 591;

3.0% w/v polyvinylpyrrolidone;

5.0% w/v polyethylene glycol-32;

0.0975% w/v sodium phosphate, dibasic;

0.5525% w/v sodium phosphate, monobasic;

0.03% w/v disodium EDTA;

0.25% w/v benzyl alcohol;

0.5% w/v glycerin;

0.015% w/v menthol; and

1.167% w/v hydroxypropyl cellulose.

In another preferred embodiment the composition comprises:

0.035% w/v brimonidine;

3.0% w/v Avicel® 591;

3.0% w/v polyvinylpyrrolidone;

5.0% w/v polyethylene glycol-32;

0.0975% w/v sodium phosphate, dibasic;

0.5525% w/v sodium phosphate, monobasic;

0.03% w/v disodium EDTA;

0.25% w/v benzyl alcohol;

0.5% w/v glycerin;

0.015% w/v menthol; and

3.0% w/v hydroxypropylmethyl cellulose.

### Method of Preparation

Preferred compositions of the present invention were prepared by first dissolving brimonidine, PEG-32, disodium EDTA, and sodium phosphate, monobasic in water. Next, polyvinylpyrrolidone was mixed into the composition until dissolved. Benzyl alcohol and glycerin were mixed into the composition prior to the completion of the dissolution of polyvinylpyrrolidone. Next, Avicel 591® was mixed in as a thickening agent along with water until the composition was about 80% of final volume. Upon completion of the dissolution of Avicel® 591, Menthol was mixed into the composition. Next, hydroxypropyl cellulose or hydroxypropylmethyl cellulose was added into the composition and left to mix for at least one hour. Finally, water was added to the final volume and pH was adjusted to 5.5.

It is clear to an expert in the art that certain substitutions to the above formulations are consistent with its effectiveness, such as replacing hydroxylpropyl cellulose with other viscosity enhancers, particularly other cellulose derivates such as hydroxylpropyl, ethyl, or methyl cellulose; use of PEG of other molecular weights, including but not limited to low molecular weight PEG 400 which may offer greater penetration of tenacious mucosal secretions more common to certain types of nasal congestion.

### EXAMPLE

Nasal-X Composition (w/v)

| | |
|---|---|
| 0.035% | brimonidine |
| 3.0% | Avicel ® 591 |
| 2.0% | hydroxypropyl cellulose |
| 3.0% | polyvinylpyrrolidone |
| 5.0% | PEG-32 |
| 0.0975% | sodium phosphate, dibasic |
| 0.5525% | sodium phosphate, monobasic |
| 0.03% | disodium EDTA |
| 0.25% | benzyl alcohol |
| 0.5% | glycerin |
| 0.00375% | menthol |
| 89.7229% | water |

Nasal-X was administered to a human subject three times a day, for 30 consecutive days. Each administration consisted of 2 sprays in each nostril. The onset of action occurred 4-6 minutes after administration and lasted from 6-8 hours. Surprisingly and unexpectedly the subject experienced no rebound effect occurred during or after the 30 day trial. Also surprisingly and unexpectedly the subject experienced no sensation of dependency, nasal pain or other irritation as a result of the administration.

What is claimed is:

1. A composition comprising from about 0.01% to about 0.04% w/v brimonidine and a vehicle selected from one or more cellulosic vehicles and polyvinylpyrrolidone or a combination thereof, wherein w/v denotes weight by volume.

2. The composition of claim 1 wherein brimonidine is at a concentration of about 0.035% w/v.

3. The composition of claim 1, wherein the one or more cellulosic vehicles are selected from carboxymethyl cellulose, microcrystalline cellulose, a mixture of carboxymethyl cellulose and microcrystalline cellulose, hydroxypropyl cellulose and hydroxpropylmethyl cellulose.

4. The composition of claim 3, wherein the vehicle comprises a mixture of carboxymethyl cellulose and microcrystalline cellulose, polyvinylpyrrolidone and hydroxypropyl cellulose.

5. The composition of claim 4 wherein the concentration of hydroxypropyl cellulose is from about 0.96% to about 1.36% w/v.

6. The composition of claim 5 wherein the concentration of hydroxypropyl cellulose is about 1.167% w/v.

7. The composition of claim 3 comprising wherein the vehicle comprises a mixture of carboxymethyl cellulose and microcrystalline cellulose, polyvinylpyrrolidone and hydroxypropylmethyl cellulose.

8. The composition of claim 7 wherein the concentration of hydroxypropylmethyl cellulose is from about 2% to about 4% w/v.

9. The composition of claim 8 wherein the concentration of hydroxypropylmethyl cellulose is about 3% w/v.

10. A method of treating a nasal condition in a subject in need thereof comprising administering to the subject a pharmaceutically effective amount of the composition of claim 1, wherein the subject experiences minimal or no rebound.

11. A composition comprising:
brimonidine at an amount of about 0.035% w/v;
3.0% w/v of a coprecipitate consisting of carboxymethyl cellulose and microcrystalline cellulose;
polyvinylpyrrolidone at an amount of about 3.0% w/v; and
hydroxypropyl cellulose at an amount of about 1.167% w/v,
wherein w/v denotes weight by volume.
